SEQUENCE LISTING (21 sequences)

SEQ ID NO: 1 - DNA, 23 bp, synthetic construct
  source 1..23: mol_type = other DNA; note = Reverse transcription primer GSP19
aagtgtcactggcatgtatacac

SEQ ID NO: 2 - DNA, 20 bp, synthetic construct
  source 1..20: mol_type = other DNA; note = Primer EX34-F
gagccccaagctgtatgtgt

SEQ ID NO: 3 - DNA, 20 bp, synthetic construct
  source 1..20: mol_type = other DNA; note = Primer EX34-R
ggacacagagtcagggggta

SEQ ID NO: 4 - DNA, 20 bp, synthetic construct
  source 1..20: mol_type = other DNA; note = Primer EX45-F
aaggggcctaagtcattgct

SEQ ID NO: 5 - DNA, 20 bp, synthetic construct
  source 1..20: mol_type = other DNA; note = Primer EX45-R
cagcagccagtgccatagta

SEQ ID NO: 6 - DNA, 22 bp, synthetic construct
  source 1..22: mol_type = other DNA; note = Primer J13-F
gcctcctcttcttcctcctctt

SEQ ID NO: 7 - DNA, 25 bp, synthetic construct
  source 1..25: mol_type = other DNA; note = Primer J13-R
ccggaacagctccccttccaccttc

SEQ ID NO: 8 - DNA, 21 bp, synthetic construct
  source 1..21: mol_type = other DNA; note = Primer H-F
tgacactggcaaaacaatgca

SEQ ID NO: 9 - DNA, 18 bp, synthetic construct
  source 1..18: mol_type = other DNA; note = Primer H-R
ggtccttttcaccagcaa

SEQ ID NO: 10 - DNA, 18 bp, synthetic construct
  source 1..18: mol_type = other DNA; note = Primer P-F
gggagatcagggaccggc

SEQ ID NO: 11 - DNA, 21 bp, synthetic construct
  source 1..21: mol_type = other DNA; note = Primer P-R
gctgtgactggaagctgggac

SEQ ID NO: 12 - DNA, 19 bp, synthetic construct
  source 1..19: mol_type = other DNA; note = Primer E-F
tgccgagtcttgctcacac

SEQ ID NO: 13 - DNA, 22 bp, synthetic construct
  source 1..22: mol_type = other DNA; note = Primer E-R
cattgactgtcgtagacaccac

SEQ ID NO: 14 - DNA, 20 bp, synthetic construct
  source 1..20: mol_type = other DNA; note = Primer FP
ggagagtctgaccaccatgc

SEQ ID NO: 15 - DNA, 20 bp, synthetic construct
  source 1..20: mol_type = other DNA; note = Primer RP
ggacacagagtcagggggta

SEQ ID NO: 16 - DNA, 20 bp, Homo sapiens
  source 1..20: mol_type = genomic DNA; note = Sequence on exon 4 of CD19 gene
taccccctgactctgtgtcc

SEQ ID NO: 17 - DNA, 25 bp, Homo sapiens
  source 1..25: mol_type = genomic DNA; note = Sequence of the junction between exon 1 and exon 3 of CD19 gene
gaaggtggaaggggagctgttccgg

SEQ ID NO: 18 - DNA, 20 bp, Homo sapiens
  source 1..20: mol_type = genomic DNA; note = Sequence at the beginning of exon 5 of CD19 gene
tactatggcactggctgctg

SEQ ID NO: 19 - DNA, 601 bp, synthetic construct
  source 1..601: mol_type = other DNA; note = sequence of the gel-cut purified product according to first-generation sequencing using the forward primer
tcctctcttcttctctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccatagcctcagccaggacctcaccatggcccctggctcccacactctggctgtcctgtggggtaccccctgact

SEQ ID NO: 20 - DNA, 1921 bp, Homo sapiens
  source 1..1921: mol_type = genomic DNA; note = CD19 NM_001770.6
aagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggcagggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaa

SEQ ID NO: 21 - DNA, 598 bp, synthetic construct
  source 1..598: mol_type = other DNA; note = sequence of the gel-cut purified product according to first-generation sequencing using the reverse primer
tctgaccaccatgccaccctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacact

(19) United States
(12) Patent Application Publication
(10) Pub. No.: US 2024/0352531 A1
(43) Pub. Date: Oct. 24, 2024

(54) METHOD FOR DETECTING CD19 EXPRESSION

(71) Applicant: UNION HOSPITAL TONGJI MEDICAL COLLEGE HUST, Wuhan City (CN)

(72) Inventors: Heng MEI, Wuhan City (CN); Chenggong LI, Wuhan City (CN); Fen ZHOU, Wuhan City (CN); Yun KANG, Wuhan City (CN)

(73) Assignee: UNION HOSPITAL TONGJI MEDICAL COLLEGE HUST, Wuhan City (CN)

(21) Appl. No.: 18/432,678

(22) Filed: Feb. 5, 2024

(30) Foreign Application Priority Data
Apr. 18, 2023 (CN) 202310413024.9

(51) Int. Cl. C12Q 1/6886

(52) U.S. Cl. CPC C12Q 1/6886; C12Q 2600/106; C12Q 2600/156; C12Q 2600/158; C12Q 2600/166

(57) ABSTRACT

The present invention belongs to the technical field of molecular biology, and specifically relates to the technical field of efficacy monitoring of a chimeric antigen receptor (CAR)-T cell therapy. The present invention provides use of a primer combination in the preparation of a product for detecting a prognostic effect of an anti-CD19 CAR-T cell therapy in a tumor, where the primer combination includes primers shown in Table 1. The present invention provides use of a primer combination in the preparation of a product for detecting an expression state of CD19 on a surface of a tumor cell in a tumor patient, where the primer combination includes primers shown in Table 1. The present invention provides a product for detecting a prognostic effect of an anti-CD19 CAR-T cell therapy in a tumor, where the product includes primers shown in Table 1 as effective components.

### CROSS REFERENCE TO RELATED APPLICATION

This patent application claims foreign priority benefits under 35 U.S.C. § 119 (a)-(d) to Chinese patent application No. 202310413024.9 filed on Apr. 18, 2023, which is hereby incorporated herein by reference in its entirety.

### REFERENCE TO SEQUENCE LISTING

A computer readable XML file entitled “HLP20231109122_seqlist”, that was created on Jan. 2, 2024, with a file size of about 26,336 bytes, contains the sequence listing for this application, has been filed with this application, and is hereby incorporated by reference in its entirety.

### TECHNICAL FIELD

The present invention belongs to the technical field of molecular biology, and in particular to efficacy monitoring of a chimeric antigen receptor (CAR)-T cell therapy.

### BACKGROUND

CD19, a transmembrane protein continuously expressed on the surface of B cells, regulates development, proliferation, and differentiation of the B cells in both B cell receptor (BCR)-dependent and -independent manners, and is also expressed in at least 95% of B-cell tumors. Chimeric antigen receptor (CAR)-T cell therapy targeting the CD19 demonstrates significant efficacy in a variety of B-cell malignancies. The CAR-T cell therapy has achieved a complete remission rate of 70% to 90% in B-cell acute lymphoblastic leukemia (B-ALL), becoming a revolutionary achievement in the field of tumor immunotherapy. However, 30% to 60% of B-ALL patients still suffer from the disease recurrence after anti-CD19 CAR-T therapy. In particular, some patients have experienced antigen-negative relapse, which limits the further application of anti-CD19 CAR-T therapy.

Among the mechanisms currently proposed for the down-regulation or loss of CD19 molecule expression on the surface of tumor cells under a pressure of the anti-CD19 CAR-T therapy, the most important one is the alternative spliceosomes of CD19 mRNA. The protein isoform produced by translation of the alternative spliceosomes lacks the extracellular segment, cell membrane localization segment, or antigenic epitope that binds to a single-chain fragment variable (scFv) of CAR-T cells targeting the CD19, thus being unable to be expressed normally in the cell membrane or recognized by the CAR-T cells. However, this mechanism needs to be further explored in more clinical anti-CD19 CAR-T cell therapy-related studies. Due to the particularity of an isoform sequence, it is necessary to establish a mature and stable detection system for the alternatively spliced isoforms. This has hindered to a certain extent the investigations of whether an alternative splicing mechanism potentially affects the outcome of ineffectiveness or relapse after anti-CD19 CAR-T therapy.

Therefore, CD19 expression in a tumor of a patient underwent the anti-CD19 CAR-T cell therapy and the proportion of alternatively spliced isoforms during the therapy are monitored and identified using molecular biology separately. This process is conducive to verifying alternative splicing in the mechanism of disease recurrence in a clinical cohort, thereby elucidating important factors influencing the efficacy of the CAR-T cell therapy.

### SUMMARY

To address the above problems, the present invention provides a method for detecting CD19 expression. The method mainly solves the problem that an antigen epitope bound by the scFv of CAR-T cells cannot be expressed normally on the cell membrane or cannot be recognized by the CAR-T cells, which makes it difficult to establish a mature and stable detection system for alternatively spliced isoforms.

To solve the problem, the present invention adopts the following technical solutions.

The present invention provides use of a primer combination in the preparation of a product for detecting a prognostic effect of an anti-CD19 CAR-T cell therapy in a tumor, where the primer combination includes one primer and four primer pairs shown in Table 1 as follows:

TABLE 1
Primer sequences

| | Primer | | | |
|---|---|---|---|---|
| Serial | pair | Orientation | Primer | |
| No. | name | of primer | name | Sequence |
| 1 | | | GSP19 | AAGTGTCACTGGCATGTATA |
| | | | | CAC (SEQ ID NO: 1) |
| 2 | EX34 | Forward | EX34-F | GAGCCCCAAGCTGTATGTGT |
| | | | | (SEQ ID NO: 2) |
| | | Reverse | EX34-R | GGACACAGAGTCAGGGGGTA |
| | | | | (SEQ ID NO: 3) |
| 3 | EX45 | Forward | EX45-F | AAGGGGCCTAAGTCATTGCT |
| | | | | (SEQ ID NO: 4) |
| | | Reverse | EX45-R | CAGCAGCCAGTGCCATAGTA |
| | | | | (SEQ ID NO: 5) |
| 4 | J13 | Forward | J13-F | GCCTCCTCTTCTTCCTCCTC |
| | | | | TT (SEQ ID NO: 6) |
| | | Reverse | J13-R | CCGGAACAGCTCCCCTTCCA |
| | | | | CCTTC (SEQ ID NO: 7) |
| 5 | H | Forward | H-F | TGACACTGGCAAAACAATGC |
| | | | | A (SEQ ID NO: 8) |
| | | Reverse | H-R | GGTCCTTTTCACCAGCAA |
| | | | | (SEQ ID NO: 9) |

In the present invention, the primer combination is to detect an expression abundance of CD19 on a surface of a tumor cell and/or to detect a proportion of alternatively spliced isoforms CD19 mRNA.

The present invention further provides use of a primer combination in the preparation of a product for detecting an expression state of CD19 on a surface of a tumor cell in a tumor patient, where the primer combination includes one primer and four primer pairs as follows:

| | Primer | | | |
|---|---|---|---|---|
| Serial | pair | Orientation | Primer | |
| No. | name | of primer | name | Sequence |
| 1 | | | GSP19 | AAGTGTCACTGGCATGTATA |
| | | | | CAC (SEQ ID NO: 1) |
| 2 | EX34 | Forward | EX34-F | GAGCCCCAAGCTGTATGTGT |
| | | | | (SEQ ID NO: 2) |
| | | Reverse | EX34-R | GGACACAGAGTCAGGGGGTA |
| | | | | (SEQ ID NO: 3) |
| 3 | EX45 | Forward | EX45-F | AAGGGGCCTAAGTCATTGCT |
| | | | | (SEQ ID NO: 4) |
| | | Reverse | EX45-R | CAGCAGCCAGTGCCATAGTA |
| | | | | (SEQ ID NO: 5) |
| 4 | J13 | Forward | J13-F | GCCTCCTCTTCTTCCTCCTC |
| | | | | TT (SEQ ID NO: 6) |
| | | Reverse | J13-R | CCGGAACAGCTCCCCTTCCA |
| | | | | CCTTC (SEQ ID NO: 7) |
| 5 | H | Forward | H-F | TGACACTGGCAAAACAATGC |
| | | | | A (SEQ ID NO: 8) |
| | | Reverse | H-R | GGTCCTTTTCACCAGCAA |
| | | | | (SEQ ID NO: 9) |

The present invention further provides a product for detecting a prognostic effect of an anti-CD19 CAR-T cell therapy in a tumor, including effective components of one primer and four primer pairs as follows:

| | Primer | | | |
|---|---|---|---|---|
| Serial | pair | Orientation | Primer | |
| No. | name | of primer | name | Sequence |
| 1 | | | GSP19 | AAGTGTCACTGGCATGTATA |
| | | | | CAC (SEQ ID NO: 1) |
| 2 | EX34 | Forward | EX34-F | GAGCCCCAAGCTGTATGTGT |
| | | | | (SEQ ID NO: 2) |
| | | Reverse | EX34-R | GGACACAGAGTCAGGGGGTA |
| | | | | (SEQ ID NO: 3) |
| 3 | EX45 | Forward | EX45-F | AAGGGGCCTAAGTCATTGCT |
| | | | | (SEQ ID NO: 4) |
| | | Reverse | EX45-R | CAGCAGCCAGTGCCATAGTA |
| | | | | (SEQ ID NO: 5) |
| 4 | J13 | Forward | J13-F | GCCTCCTCTTCTTCCTCCTC |
| | | | | TT (SEQ ID NO: 6) |
| | | Reverse | J13-R | CCGGAACAGCTCCCCTTCCA |
| | | | | CCTTC (SEQ ID NO: 7) |
| 5 | H | Forward | H-F | TGACACTGGCAAAACAATGC |
| | | | | A (SEQ ID NO: 8) |
| | | Reverse | H-R | GGTCCTTTTCACCAGCAA |
| | | | | (SEQ ID NO: 9) |

Regarding the primer (pair) combinations above: the primer pairs 1 to 4 are designed for the exons of a CD19 gene, and the 5 splicing sites are designed based on a structure of the alternative spliceosome of a CD19 RNA. The alternative spliceosome of the CD19 RNA mainly includes a full-length CD19 RNA, an alternatively spliced isoform with a second exon deleted (Δex2 alternative spliceosome), and a Δex5-6 alternative spliceosome with the deletion of exons 5 to 6. Since the structures of different alternative spliceosomes vary, it is necessary to find unique and common RNA sequence sites of the different alternative spliceosomes to design primers. Furthermore, the primers can amplify and quantify these alternatively spliced isoforms only after primer specificity has been verified by National Center for Biotechnology Information (5NCBI BLAST. In addition, primer 5 is used to amplify a reference gene HPRT1. Since the expression level of CD19 is relatively low, the reference gene is required to have low expression in cells to increase the accuracy of relative quantification. The present invention improves the accuracy through a suitable design. By calculating ct values amplified by the reference primer and other primers, relative expression levels of the CD19 and different alternatively spliced isoforms thereof are finally calculated. Therefore, all the 5 primer (pairs) are essential. The forward and reverse primers are designed on different exons, which is helpful for distinguishing whether a product is generated by reverse transcription or is an amplified DNA fragment in gel electrophoresis. This can be used as an alternative only if other primers with different sequences are designed for a same site and can successfully amplify a product corresponding to the RNA sequence. Other alternatives need to overcome the specificity of primer design, the efficiency of amplification reaction, and the low expression abundance of CD19 gene. Due to different primer designs, the amplification results can inevitably lead to different first-generation sequencing results. Meanwhile, any deliberate addition of other primers to the primer combination of the present invention should be regarded as adopting the solution of the present invention, provided that there are no new and unexpected effects after adding the other primers.

The present invention further provides a method for detecting an expression state of CD19 in a tumor patient underwent an anti-CD19 CAR-T cell therapy, including the following steps:
S1, extracting an RNA from a bone marrow sample and synthesizing a cDNA:
   extracting a total RNA from the bone marrow sample of a patient, and determining a concentration and an A260/280 value of the total RNA; and
   subjecting the total RNA to reverse transcription using a specific primer GSP19 of a CD19 gene to obtain a cDNA stock solution, and determining a concentration and an A260/280 value of the cDNA stock solution, where the specific primer GSP19 has a sequence of 5′-AAGTGTCACTGGCATGTATACAC-3′ (SEQ ID NO:1);
S2, conducting polymerase chain reaction (PCR) amplification and quantitative real-time PCR (qRT-PCR):
   qRT-PCR: mixing each component, primers, and ddH₂O in a reaction system by a kit in proportion to obtain a mixture, centrifuging the mixture, aliquoting a resulting centrifuged mixture into a well plate for the qRT-PCR, adding a template cDNA into the well plate, centrifuging the well plate, conducting the PCR amplification and the qRT-PCR (where a reaction procedure directly refers to prior art, or be conducted according to the corresponding kit parameters), and monitoring a result of the qRT-PCR; where
   in the reaction system: CD19junct1-3 primers (primer pair J13) have a final concentration of 200 nM, and CD19exon3-4 primers (primer pair EX34) and the CD19exon4-5 primers (primer pair EX45) each have a final concentration of 100 nM, (for example, primers synthesized by Tsingke Biotech can be used after dilution to 10 μM with sterile ddH₂O according to the instructions), and
   sequences of the primer pairs include:

| | Amplific- | Primer | Orient- | | |
|---|---|---|---|---|---|
| Serial | ation | pair | ation of | Primer | |
| No. | region | name | primer | name | Sequence |
| 6 | hPAX5 | P | Forward | P-F | GGGAGATCAGGG |
| | | | | | ACCGGC (SEQ |
| | | | | | ID NO: 10) |
| | | | Reverse | P-R | GCTGTGACTGGA |
| | | | | | AGCTGGGAC |
| | | | | | (SEQ ID NO: |
| | | | | | 11) |
| 7 | hEBF1 | E | Forward | E-F | TGCCGAGTCTTG |
| | | | | | CTCACAC (SEQ |
| | | | | | ID NO: 12) |
| | | | Reverse | E-R | CATTGACTGTCG |
| | | | | | TAGACACCAC |
| | | | | | (SEQ ID NO: |
| | | | | | 13) |
| 1 | CD19- | EX34 | Forward | EX34-F | GAGCCCCAAGCT |
| | exon3-4 | | | | GTATGTGT |
| | | | | | (SEQ ID NO: |
| | | | | | 2) |
| | | | Reverse | EX34-R | GGACACAGAGTC |
| | | | | | AGGGGGTA |
| | | | | | (SEQ ID NO: |
| | | | | | 3) |
| 2 | CD19- | EX45 | Forward | EX45-F | AAGGGGCCTAAG |
| | exon4-5 | | | | TCATTGCT |
| | | | | | (SEQ ID NO: |
| | | | | | 4) |
| | | | Reverse | EX45-R | CAGCAGCCAGTG |
| | | | | | CCATAGTA |
| | | | | | (SEQ ID NO: |
| | | | | | 5) |
| 3 | CD19- | J13 | Forward | J13-F | GCCTCCTCTTCT |
| | junct1-3 | | | | TCCTCCTCTT |
| | | | | | (SEQ ID NO: |
| | | | | | 6) |
| | | | Reverse | J13-R | CCGGAACAGCTC |
| | | | | | CCCTTCCACCTT |
| | | | | | C (SEQ ID |
| | | | | | NO: 7) |
| 4 | HPRT1 | H | Forward | H-F | TGACACTGGCAA |
| | | | | | AACAATGCA |
| | | | | | (SEQ ID NO: |
| | | | | | 8) |
| | | | Reverse | H-R | GGTCCTTTTCAC |
| | | | | | CAGCAA (SEQ |
| | | | | | ID NO: 9) |

In the present invention, the qRT-PCR program includes: 1) initial denaturation at 95° C. for 5 min, 2) denaturation at 95° C. and annealing at 60° C., 40 cycles, 3) melt curve, denaturation at 95° C. for 15 s, annealing at 60° C., and denaturation at 95° C. The conditions similar to the reaction procedures of the present invention under the premise of similar purposes should be regarded as within the scope of the present invention, and other conventional reaction procedures should also be regarded as equivalent.
S3, reading CT values of multiple duplicates of an amplification curve of each of the amplification primer pairs according to an effective amplification result of the qRT-PCR, and removing reactions with a CT value of greater than 35; averaging the CT values of the multiple duplicates of the amplification curve of each of the amplification primer pairs (referring to formula I), calculating Act according to formulas I and II with the internal reference HPRT1 (primer pair H) or the amplification curve of the EX34 primer pair as a control; and calculating a level of a target mRNA of the CD19 by a 2-Act relative quantification method (the prior art) according to formulas III and IV; where
   formula I: MEAN ct(X)=(ct(duplicate 1×)+ct(duplicate 2×)+ . . . +ct(duplicate nX))/n (for example: MEAN ct(internal reference)=(ct(duplicate 1 internal reference)+ct(duplicate 2 internal reference)+ . . . +ct(duplicate n internal reference))/n),
   formula II: Δct(target mRNA)=MEAN ct(target mRNA)−MEAN ct(internal reference); formula III: relative expression level (fold-change)=2\hat{ }(−Δct(target mRNA)), and
   formula IV: MEAN ct(target mRNA)=(ct(duplicate 1 target RNA)+ct(duplicate 2 target RNA)+ . . . +ct(duplicate n target RNA))/n; it should be noted that, on the premise that the aforementioned parameters are known, specific steps for calculating a target mRNA level of the CD19 in combination with the 2-Δct relative quantification method are existing technologies, and the calculation can also be conducted using instruments, which are not repeated in detail;
   qRT-PCR results can also be relatively quantified to determine an expression proportion of different alternatively spliced isoforms in mRNA of the CD19 (specific methods refer to the prior art).

In some cases, the method for detecting an expression state of CD19 in a tumor patient treated by an anti-CD19 CAR-T cell therapy further includes the following steps:
S4, mixing a CD19exon1-4 amplification primer pair in the reaction system to obtain a mixture, centrifuging the mixture, aliquoting a resulting centrifuged mixture into a tube strip, and adding a template cDNA in each tube of the tube strip to allow PCR amplification; where
sequences of the CD19exon1-4 amplification primer pair are:

| | FP: |
|---|---|
| (SEQ ID NO: 14) | |
| | GGAGAGTCTGACCACCATGC, |
| | RP: |
| (SEQ ID NO: 15) | |
| | GGACACAGAGTCAGGGGGTA; |

the template cDNA is prepared from the cDNA stock solution in step S1;
the reaction system includes: the CD19exon1-4 amplification primer pair with a final concentration of 400 nM, 2 μL to 5 μL of the cDNA stock solution synthesized in step S1 as the template cDNA, and a 2×Taq Plus MasterMix DNA polymerase; and
   the PCR amplification includes: initial denaturation at 95° C.; denaturation at 95° C., annealing at 60° C., and extension at 72° C., 35 cycles; and final extension at 72° C.; and
S5: subjecting a product obtained from the PCR amplification in step S4 to 1.5% agarose gel electrophoresis, determining a band and cutting a gel containing the band to allow purification, conducting bidirectional first-generation sequencing using the forward amplification primer and the reverse amplification primer in S2, and analyzing an obtained result. After electrophoresis, the position of a product band is determined based on the marker, a single bright band is cut off with a blade in a Blue Light LED Transilluminator, and stored in an EP tube. The cut band and corresponding reaction primers are subjected to subsequent purification and bidirectional first-generation sequencing to obtain results. Applied Biosystems (ABI) data obtained by bidirectional sequencing are compared with a CD19 transcript in NCBI to determine whether the product is an alternative spliceosome.

In some cases, the reaction system in step S2 further includes an hPAX5 amplification primer pair (primer pair P) and a hEBF1 amplification primer pair (primer pair E). The primer pairs P and E are mainly used to determine a transcription level of the CD19 gene, The transcription level of the CD19 gene is determined, and then the alternative spliceosome of CD19 is determined. In the reaction system of step S2, the primer pair P, the primer pair E, and the primer pair J13 each have a final concentration of 200 nM.

Detecting the expression abundance of CD19 on the surface of tumor cells and/or detecting the proportion of alternatively spliced isoforms of CD19 mRNA is intended to detect the expression abundance of CD19 on the surface of a tumor cell and/or to detect the proportion of alternatively spliced isoforms in CD19 mRNA.

The embodiments of the present invention has following beneficial effects:

In the present invention, the CD19 expression in a tumor of a patient underwent the anti-CD19 CAR-T cell therapy and the proportion of alternatively spliced isoforms during the therapy are monitored and identified using molecular biology separately. Moreover, alternative splicing in the mechanism of disease recurrence is verified in a clinical cohort, thereby improving a monitoring approach for the efficacy of the CAR-T cell therapy.

### BRIEF DESCRIPTION OF THE DRAWINGS

FIG. 1 shows a flow chart;

FIG. 2 to FIG. 3 show linear and logarithmic amplification curves using 6 primer pairs;

FIG. 4 shows melt curves using 6 primer pairs;

FIG. 5 shows amplification results using CD19-related primers with HPRT1 as an internal reference;

FIG. 6 shows amplification results using CD19 primers with CD19exon3-4 as an internal reference;

FIG. 7 shows a band of the amplification product using CD19 exon1-4 primer;

FIG. 8 shows alignment of sequence of gel-cut purified product according to bidirectional first-generation sequencing;

FIG. 9 shows alignment of the sequence of the gel-cut purified product according to first-generation sequencing using the forward primer; and

FIG. 10 shows alignment of the sequence of the gel-cut purified product according to first-generation sequencing using the reverse primer.

### DETAILED DESCRIPTION OF THE EMBODIMENTS

The present invention will be further described below in conjunction with specific research projects:

### I. Main Experimental Materials:

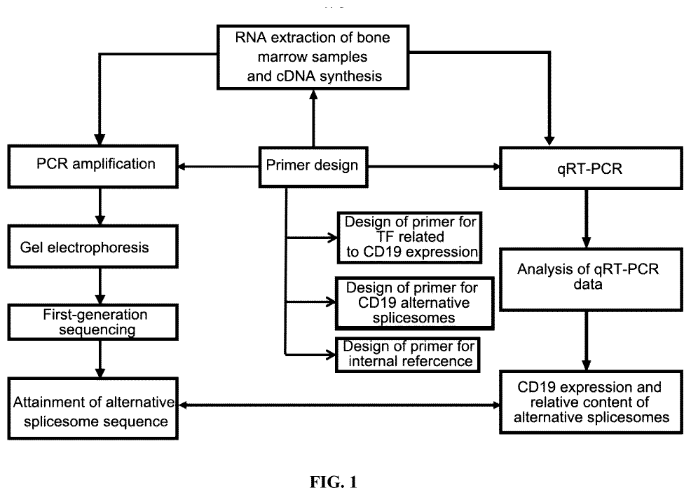

Cwbio CW0581M ultrapure RNA extraction kit was purchased from CoWin Biotech Co., Ltd;
HiScript® III 1st Strand cDNA Synthesis (+gDNA wiper) kit, AceQ® qPCR SYBR Green Master Mix kit, and 2×Taq Plus Master Mix PCR reaction kit were purchased from Nanjing Vazyme Biotech Co., Ltd.;
main experimental instruments:
small centrifuge
high-speed refrigerated centrifuge, thermofic
Nanodrop 8000, thermofic
C1000 Touch™ thermal Cycler PCR instrument, BIO-RAD
ABI 7500fast Real time PCR systems, qRT-PCR instrument
Analysis software: Design & Analysis Software version 2.6.0, Copyright©2021 Thermo Fisher Scientific; SnapGene@ version 6.0.2; GraphPad Prism version 9.0.2.
   II. The technical process diagram of this research was shown in FIG. 1, mainly including the following steps:

1. Extraction of RNA from bone marrow sample and synthesis of cDNA
(1) Total RNA from bone marrow samples was extracted with Cwbio CW0581M ultrapure RNA extraction kit according to kit instructions. The concentration and A260/280 value of total RNA were determined by NanoDrop8000 microvolume UV spectrophotometer in RNA mode.
(2) The total RNA was reverse-transcribed using Vazyme HiScript® III 1st Strand cDNA Synthesis (+gDNA wiper) kit into cDNA for subsequent qRT-PCR with Oligo (dT) 20VN and Random hexamers according to the kit instructions. After the specific primer GSP19 of the CD19 gene was reverse-transcribed into cDNA for subsequent PCR, the cDNA concentration and A260/280 value were measured by NanoDrop 8000 micro-volume UV spectrophotometer in ssDNA mode.

2. The CD19 gene-specific reverse transcription primer GSP19 was designed based on a predicted sequence of human CD19 gene mRNA published by NCBI (Genbank accessionNo.NM_001178098.2; NM_001770.6; NM_001385732.1) to reversely generate the cDNA; the primer pairs EX34 and J13 for directly or indirectly identifying the deletion of exon 2 of the CD19 gene or the primer pair EX45 for deleting the alternative spliceosome of exons 5 to 6 were designed. The primer pair P and E were designed based on the human PAX5 gene sequence (Genbank accession No.NM_016734.3) and EBF1 gene sequence (Genbank accession No. NM_024007.5) published by NCBI. The primer pair H was designed based on the human HPRT1 gene sequence published by NCBI (Genbank accession NM_000194.3):
(1) the sequence of the CD19 gene-specific reverse transcription primer GSP19 was:

| (SEQ ID NO: 1) | |
|---|---|
| | 5′-AAGTGTCACTGGCATGTATACAC-3′; |

(2) the primer pair EX34 sequences for amplifying the third exon and the fourth exon were:

| | forward primer: |
|---|---|
| (SEQ ID NO: 2) | |
| | 5′-GAGCCCCAAGCTGTATGTGT-3′, |
| | and |
| | reverse primer: |
| (SEQ ID NO: 3) | |
| | 5′-GCCCAATACGACCAAATCCGT-3′; |

(3) the primer pair EX45 sequences for amplifying the fourth exon and the fifth exon were:

| | forward primer: |
|---|---|
| (SEQ ID NO: 4) | |
| | 5′-AAGGGGCCTAAGTCATTGCT-3′, |
| | and |
| | reverse primer: |
| (SEQ ID NO: 5) | |
| | 5′-CAGCAGCCAGTGCCATAGTA-3′; |

(4) the primer pair J13 sequences for amplifying a junction of exon 1 and exon 3 were:

| | forward primer: |
|---|---|
| (SEQ ID NO: 6) | |
| | 5′-GCCTCCTCTTCTTCCTCCTCTT-3′, |
| | and |
| | reverse primer: |
| (SEQ ID NO: 7) | |
| | 5′-CCGGAACAGCTCCCCTTCCACCTTC-3′; |

(5) the primer pair H sequences for the internal reference gene HPRT1 were:

| | forward primer: |
|---|---|
| (SEQ ID NO: 8) | |
| | 5′-TGACACTGGCAAAACAATGCA-3′, |
| | and |
| | reverse primer: |
| (SEQ ID NO: 9) | |
| | 5′-GGTCCTTTTCACCAGCAA-3′. |

The primer pair CD19exon3-4 were used to detect all mRNAs of the human CD19 gene, including all full-length and alternatively splicesomes, where the forward primer was designed based on the sequence of exon 3 (5′-GAGCCCCAAGCTGTATGTGT-3′, SEQ ID NO: 2) of the relatively conserved region of the CD19 gene mRNA, and the reverse primer was designed based on the sequence (5′-TACCCCCTGACTCTGTGTCC-3′, SEQ ID NO: 16) on exon 4. The primer pair CD19Junct1-3, which specifically recognizes the Δex2 alternative spliceosome, were used to detect the Δex2 alternative spliceosome, where the reverse primer was designed based on the junction between exon 1 and exon 3 (5′-GAAGGTGGAAGGGGAGCTGTTCCGG-3′, SEQ ID NO: 17) after deleting 261 base pair (bp) of all exon 2 of the human CD19 gene sequences at the CD19 gene mRNA level, and the forward primer was designed baed on the sequence of exon 1. The primer pair CD19junct1-3 could only amplify the spliceosome with the deletion of exon 2, and could avoid interference from other known or unknown CD19 gene alternative spliceosomes. The primer pair CD19exon4-5 were used to detect the Δex5-6 alternative spliceosome with deletions in exons 5 to 6 of the human CD19 gene, where the reverse primer was designed based on the sequence at the beginning of exon 5 of the CD19 gene (5′-TACTATGGCACTGGCTGCTG-3′, SEQ ID NO: 18), and the forward primer was designed based on the sequence of exon 4. The primer pair CD19exon4-5 could amplify CD19 mRNA containing exon 5; a relative content of the Δex5-6 alternative spliceosome could be calculated by subtracting the amplification result of CD19exon4-5 from the amplification result of CD19exon3-4. By analyzing a relative expression abundance of the CD19 gene in the bone marrow samples, the internal reference gene HPRT1 suitable as a low-expression gene was selected.

3. The reaction system was calculated with the cDNA in step 1 as a template (n=3) and qRT-PCR amplification was conducted.

In the qRT-PCR system, the primer pair P, primer pair E, and primer pair J13 each had a final concentration of 200 nM, and the primer pair EX34 and primer pair EX45 each had a final concentration of 100 nM. The primers synthesized by Tsingke Biotech were diluted to 10 μM with sterile ddH₂O according to the instructions for later use; a template cDNA was the cDNA stock solution synthesized in the previous step, and its final concentration in the qRT-PCR system was 100 ng/μL. Each component in Vazyme AceQ® qPCR SYBR Green Master Mix kit (2×AceQ qPCR SYBR Green Master Mix and 50×ROX Reference Dye 2), upstream and downstream primers, and ddH₂O in the reaction system were mixed in advance according to the proportion, centrifuged momentarily, and aliquoted into a 96-well plate dedicated for qRT-PCR, and the template cDNA was added separately. The above operations should be conducted in the dark as much as possible, and liquid in the pipette tip should be drained as much as possible when samples were added. Finally, the 96-well plate was centrifuged at 2,500 rpm for 3 min to ensure that there were no bubbles in the reaction solution before qRT-PCR was conducted.

(1) Upstream and downstream primers: amplification primers for qRT-PCR;

| | Amplific- | Primer | Orient- | | |
|---|---|---|---|---|---|
| Serial | ation | pair | ation of | Primer | |
| No. | region | name | primer | name | Sequence |
| 6 | hPAX5 | P | Forward | P-F | GGGAGATCAGGG |
| | | | | | ACCGGC (SEQ |
| | | | | | ID NO: 10) |
| | | | Reverse | P-R | GCTGTGACTGGA |
| | | | | | AGCTGGGAC |
| | | | | | (SEQ ID NO: |
| | | | | | 11) |
| 7 | hEBF1 | E | Forward | E-F | TGCCGAGTCTTG |
| | | | | | CTCACAC (SEQ |
| | | | | | ID NO: 12) |
| | | | Reverse | E-R | CATTGACTGTCG |
| | | | | | TAGACACCAC |
| | | | | | (SEQ ID NO: |
| | | | | | 13) |
| 1 | CD19- | EX34 | Forward | EX34-F | GAGCCCCAAGCT |
| | exon3-4 | | | | GTATGTGT |
| | | | | | (SEQ ID NO: |
| | | | | | 2) |
| | | | Reverse | EX34-R | GGACACAGAGTC |
| | | | | | AGGGGGTA |
| | | | | | (SEQ ID NO: |
| | | | | | 3) |
| 2 | CD19- | EX45 | Forward | EX45-F | AAGGGGCCTAAG |
| | exon4-5 | | | | TCATTGCT |
| | | | | | (SEQ ID NO: |
| | | | | | 4) |
| | | | Reverse | EX45-R | CAGCAGCCAGTG |
| | | | | | CCATAGTA |
| | | | | | (SEQ ID NO: |
| | | | | | 5) |
| 3 | CD19- | J13 | Forward | J13-F | GCCTCCTCTTCT |
| | junct1-3 | | | | TCCTCCTCTT |
| | | | | | (SEQ ID NO: |
| | | | | | 6) |
| | | | Reverse | J13-R | CCGGAACAGCTC |
| | | | | | CCCTTCCACCTT |
| | | | | | C (SEQ ID |
| | | | | | NO: 7) |
| 4 | HPRT1 | H | Forward | H-F | TGACACTGGCAA |
| | | | | | AACAATGCA |
| | | | | | (SEQ ID NO: |
| | | | | | 8) |
| | | | Reverse | H-R | GGTCCTTTTCAC |
| | | | | | CAGCAA (SEQ |
| | | | | | ID NO: 9) |

(2) Reaction program:
Stage1: initial denaturation at 95° C. for 5 min;
Stage2: denaturation at 95° C. for 10 s and annealing at 60° C. for 30 s, 40 cycles;
Stage3: melt curve, denaturation at 95° C. for 15 s, annealing at 60° C. for 60 s, and 95° C. for 15 s.

4. Software operations:
(1) ABI 7500fast software was run, and a new experiment was created and named;
(2) an instrument model was selected (ABI 7500fast instrument 96wells);
(3) 2-ΔΔct was selected as the experiment type;
(4) SYBR@ Green Reagents was selected as the reagent type;
(5) an standard operating mode was selected;
(6) for Plate Setup, the gene (Target) and sample (Sample) positions were named and arranged;
(7) a reference gene was selected as HPRT1;
(8) the Run Method interface under Setup was entered, and reaction conditions were set up; and
(9) the program was run.

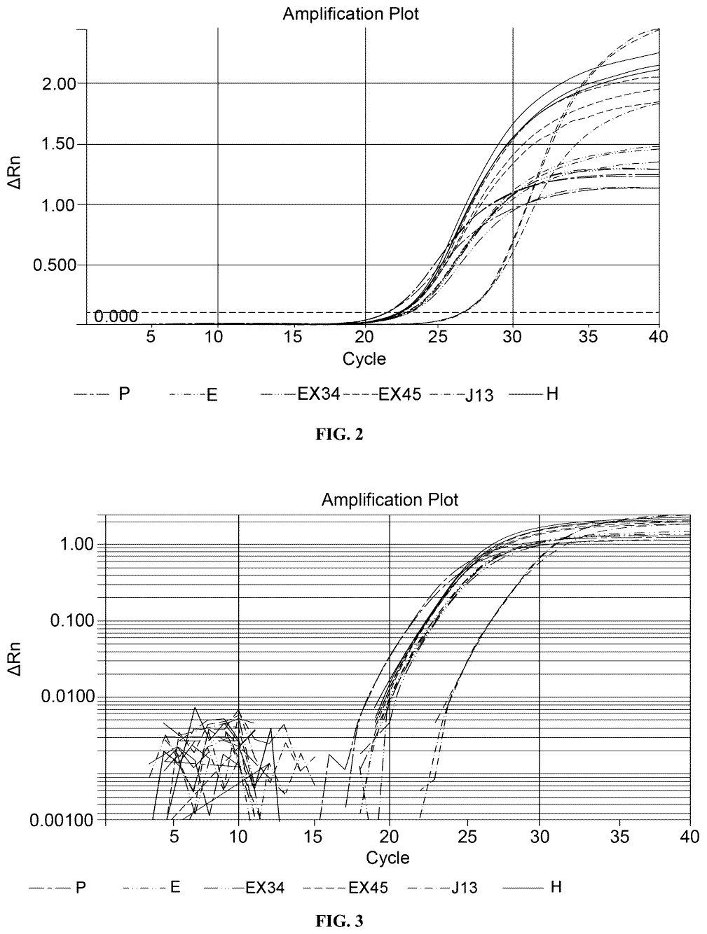

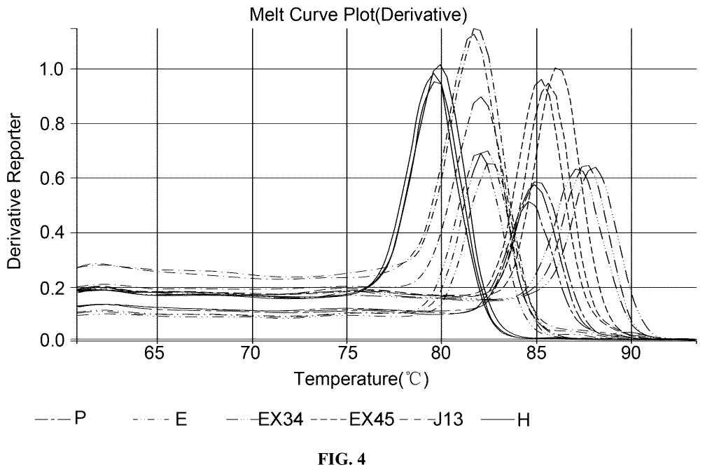

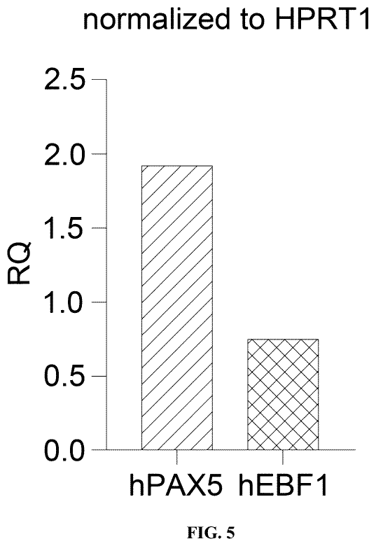

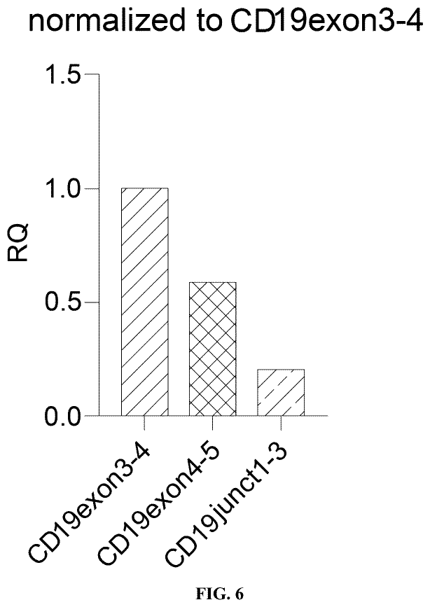

5. According to the results of qRT-PCR, the amplification curve (FIG. 2 and FIG. 3) and the melt curve (FIG. 4) of the alternative spliceosome deleted in the second exon and exons 5 to 6 of the CD19 gene and the internal reference gene HPRT1 were analyzed. In FIG. 2, the amplification curves of each gene are sigmoid and reach the plateau phase, indicating effective amplification; in FIG. 4, the melt curve of each gene has a single peak, indicating that the amplification product was specific, free of primer dimers and other non-specific amplification, and could be used for subsequent analysis of the results. The CT values of multiple duplicates of the amplification curve of each of the primer pairs, and reactions with CT value >35 were removed. After averaging, the internal reference HPRT1 was used as a control or the amplification curve of the CD19exon3-4 was used as a control to calculate Act. The levels of all CD19 mRNAs could be calculated using the 2-Δct relative quantification method. The qRT-PCR results could also be relatively quantified to accurately and quickly analyze the expression proportion of different alternative spliceosomes of mRNA in CD19 (FIG. 5 to FIG. 6).

6. PCR amplification was conducted using the cDNA generated by reverse transcription PCR with the CD19 gene-specific primer GSP19 in step 2.

In the PCR system, the final concentration of the CD19exon1-4 primer pair was 400 nM, and primers synthesized by Tsingke Biotech were diluted to 10 μM with sterile ddH₂O according to the instructions and could be used; the template cDNA was 2 μL to 5 μL of the cDNA stock solution synthesized in the previous step; and a 2×TaqPlus Master Mix DNA polymerase was used. In advance, each component, upstream and downstream primers, and ddH₂O in the reaction system were mixed according to the proportion, centrifuged briefly, and aliquoted into a 0.4 mL 8-tube strip, and the template cDNA was added separately. The reaction program included: initial denaturation at 95° C. for 3 min; denaturation at 95° C. for 15 s, annealing at 60° C. for 15 s, and extension at 72° C. for 52 s, 35 cycles; and final extension at 72° C. for 5 min.

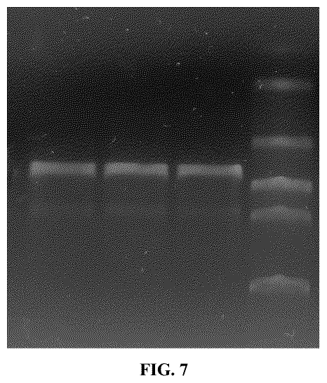

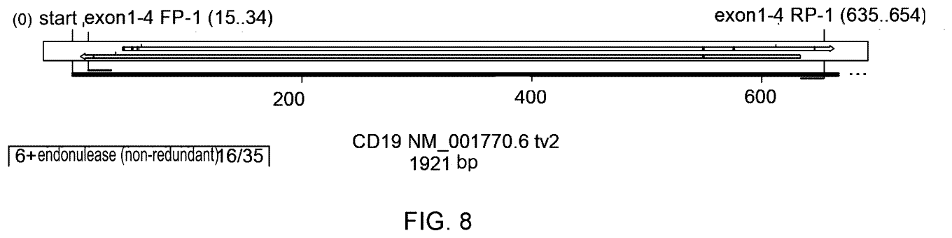

7. A 1.5% agarose gel was prepared to allow electrophoresis of a PCR product obtained in step 6 at 120 V for 30 min. The bands were observed (FIG. 7). The bidirectional first-generation sequencing was conducted using the forward amplification primer and the reverse amplification primer, and an obtained result was analyzed (FIG. 8 to FIG. 10).

### III. Research Results

The linear amplification curve shown in FIG. 2: qRT-PCR (N=3) was conducted on the cDNA of the patient's PBMC using the 6 primer pairs shown in the figure. It was seen that the linear amplification curve was smooth and there was desirable repeatability between duplicates.

The logarithmic amplification curve shown in FIG. 3: qRT-PCR (N=3) was conducted on the cDNA of the patient's PBMC using the 6 primer pairs shown in the figure. It was seen that the logarithmic amplification curve was normal and there was desirable repeatability between duplicates.

The melt curve shown in FIG. 4: qRT-PCR (N=3) was conducted on the cDNA of the patient's PBMC using the 6 primer pairs shown in the figure. It was seen that the melt curves of the 6 pairs of primers were in well shape, with no single peak before and after, the melting temperature was 80° C. to 88° C., and there was desirable repeatability between duplicates.

The amplification results using CD19-related primers, as shown in FIG. 5, when HPRT1 was used as an internal reference: qRT-PCR was conducted on cDNA of patient's PBMC (N=3). The HPRT1 was used as the internal reference, the ct values amplified by hPAX5 and hEBF1 primers were calculated and analyzed by the 2Δct method to obtain relative expression levels.

The amplification results of each CD19 primer, as shown in FIG. 6, when using CD19exon3-4 as the internal reference: cDNA of patient PBMC was subjected to qRT-PCR (N=3). CD19exon3-4 was used as the internal reference, and the ct values amplified by EX45 and J13 primer pairs were calculated and analyzed by the 2Δct method to obtain relative expression levels. CD19exon4-5 represented the relative proportion of all CD19 RNAs except the Δex5-6 alternatively spliced isoforms; the CD19junct1-3 represented the proportion of Δex2 alternatively spliced isoforms relative to all CD19 RNAs.

The CD19exon1-4 primer pair amplification product band is shown in FIG. 7: PCR was conducted on the cDNA of the patient's PBMC with the CD19exon1-4 primer pair (N=3), and agarose gel electrophoresis was conducted to obtain a darker band and a lighter band. According to the alignment with the marker, the darker band was located between 500 bp and 750 bp, and the lighter band was located at 250 bp.

FIG. 8 showed the alignment of bidirectional first-generation sequencing of gel-cut purified products: PCR was conducted on the cDNA of the patient's PBMC with CD19exon1-4 primer pair, and the PCR product was subjected to agarose gel electrophoresis and bidirectional first-generation sequencing. After gel cutting and purification of the darker band, the forward sequencing sequence and reverse sequencing sequence were obtained and aligned to the CD19 transcript NM_001770.6 amplified by the primers, and it was found that they were highly consistent.

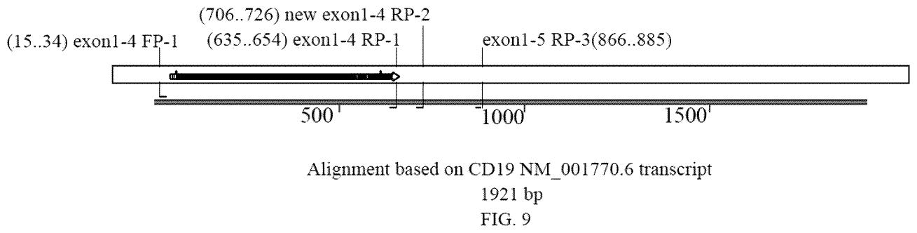

FIG. 9 showed the alignment of the first-generation sequencing of the gel-cut purified product using the forward primer: the sequence of the gel-cut purified product sequenced using the forward primer was aligned carefully to that of the amplification product by the CD19exon1-4 primer pair, indicating that the sequences were highly matched.

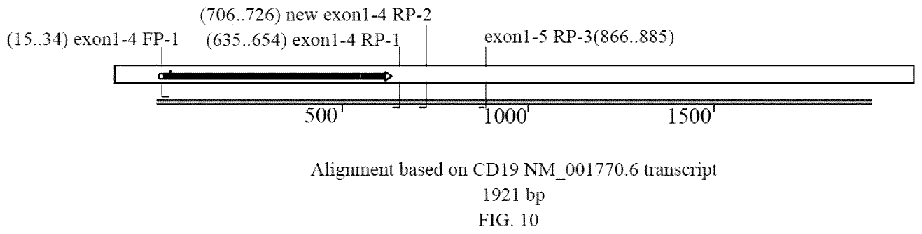

FIG. 10 showed the alignment of the first-generation sequencing of the gel-cut purified product using the reverse primer: the sequence of the gel-cut purified product sequenced using the reverse primer was aligned carefully to that of the amplification product of the CD19exon1-4 primer pair, indicating that the sequences were highly matched.

The experiment of the present invention is about the expression of multiple alternative splicing transcripts of human CD19 and its related gene transcripts. After alignment and calculation, it was found that the transcripts of the CD19 gene were mainly full-length mRNA with the highest expression level, and the spliceosome with deletion of the second exon and the spliceosome with deletion of exons 5 and 6 accounted for a lower proportion in this sample. Vertical observation of changes in the proportion of CD19 alternative spliceosomes in samples before and after relapse after CD19 CAR-T therapy could provide a means for subsequent verification of alternative spliceosomes as one of the important mechanisms of CD19-negative relapse.

It will be clear to those skilled in the art that various modifications to the above embodiments can be made without departing from the general spirit and concept of the present invention. These modifications shall all fall within the protection scope of the present invention. The claimed protection schemes of the present invention shall be determined by the claims.

1. A method of using a primer combination in the preparation of a product for detecting a prognostic effect of an anti-CD19 chimeric antigen receptor (CAR)-T cell therapy in a tumor, wherein the primer combination comprises a primer and 4 primer pairs as follows:

| | Primer | | |
|---|---|---|---|
| | or | Orient- | |
| | primer | ation | |
| Serial | pair | of | |
| Number | name | primer | Sequence |
| 1 | Reverse | | AAGTGTCACTGGCATGTATA |
| | tran- | | CAC (SEQ ID NO: 1) |
| | scription | | |
| | primer | | |
| | GSP19 | | |
| 2 | Primer | Forward | GAGCCCCAAGCTGTATGTGT |
| | pair | | (SEQ ID NO: 2) |
| | EX34 | | |
| | | Reverse | GGACACAGAGTCAGGGGGTA |
| | | | (SEQ ID NO: 3) |
| 3 | Primer | Forward | AAGGGGCCTAAGTCATTGCT |
| | pair | | (SEQ ID NO: 4) |
| | EX45 | | |
| | | Reverse | CAGCAGCCAGTGCCATAGTA |
| | | | (SEQ ID NO: 5) |
| 4 | Primer | Forward | GCCTCCTCTTCTTCCTCCTC |
| | pair | | TT (SEQ ID NO: 6) |
| | J13 | | |
| | | Reverse | CCGGAACAGCTCCCCTTCCA |
| | | | CCTTC (SEQ ID NO: 7) |
| 5 | Primer | Forward | TGACACTGGCAAAACAATGC |
| | pair H | | A (SEQ ID NO: 8) |
| | | Reverse | GGTCCTTTTCACCAGCAA |
| | | | (SEQ ID NO: 9) |

2. The method according to claim 1, wherein the primer combination is to detect an expression abundance of CD19 on a surface of a tumor cell and/or to detect a proportion of alternatively spliced isoforms in CD19 mRNA.

3. A product for detecting a prognostic effect of an anti-CD19 CAR-T cell therapy in a tumor, comprising effective components of 5 primers as follows:

| | Primer | | |
|---|---|---|---|
| | or | Orient- | |
| | primer | ation | |
| Serial | pair | of | |
| Number | name | primer | Sequence |
| 1 | Reverse | | AAGTGTCACTGGCATGTATA |
| | tran- | | CAC (SEQ ID NO: 1) |
| | scription | | |
| | primer | | |
| | GSP19 | | |
| 2 | Primer | Forward | GAGCCCCAAGCTGTATGTGT |
| | pair | | (SEQ ID NO: 2) |
| | EX34 | | |
| | | Reverse | GGACACAGAGTCAGGGGGTA |
| | | | (SEQ ID NO: 3) |
| 3 | Primer | Forward | AAGGGGCCTAAGTCATTGCT |
| | pair | | (SEQ ID NO: 4) |
| | EX45 | | |
| | | Reverse | CAGCAGCCAGTGCCATAGTA |
| | | | (SEQ ID NO: 5) |
| 4 | Primer | Forward | GCCTCCTCTTCTTCCTCCTC |
| | pair | | TT (SEQ ID NO: 6) |
| | J13 | | |
| | | Reverse | CCGGAACAGCTCCCCTTCCA |
| | | | CCTTC (SEQ ID NO: 7) |
| 5 | Primer | Forward | TGACACTGGCAAAACAATGC |
| | pair H | | A (SEQ ID NO: 8) |
| | | Reverse | GGTCCTTTTCACCAGCAA |
| | | | (SEQ ID NO: 9) |

4. A method for detecting an expression state of CD19 in a tumor patient underwent an anti-CD19 CAR-T cell therapy, comprising the following steps:
S1, extracting RNA from a bone marrow sample and synthesizing cDNA:
extracting total RNA from the bone marrow sample of a patient, and determining a concentration and an A260/280 value of the total RNA; and
subjecting the total RNA to reverse transcription using a specific primer GSP19 of 5′-AAGTGTCACTGGCATGTATACAC-3′ (SEQ ID NO: 1) to obtain a cDNA stock solution, and determining a concentration and an A260/280 value of the cDNA stock solution;
S2, conducting polymerase chain reaction (PCR) amplification and quantitative real-time PCR (qRT-PCR):
mixing primer pair J13, primer pair EX34, and primer pair EX45 in a reaction system in a kit to obtain a mixture, centrifuging the mixture, aliquoting the mixture into a well plate for the qRT-PCR, adding a template cDNA into the well plate, centrifuging the well plate, conducting the PCR amplification, and monitoring a result of the qRT-PCR; wherein
the template cDNA is prepared from the cDNA stock solution in step S1;
sequences of amplification primer pairs are shown in the following:

| | Amplific- | Primer | Orient- | | |
|---|---|---|---|---|---|
| Serial | ation | pair | ation of | Primer | |
| Number | region | name | primer | name | Sequence |
| 2 | CD19- | EX34 | Forward | EX34-F | GAGCCCCAAGCTG |
| | exon3-4 | | | | TATGTGT (SEQ |
| | | | | | ID NO: 2) |
| | | | Reverse | EX34-R | GGACACAGAGTCA |
| | | | | | GGGGGTA (SEQ |
| | | | | | ID NO: 3) |
| 3 | CD19- | EX45 | Forward | EX45-F | AAGGGGCCTAAGT |
| | exon4-5 | | | | CATTGCT (SEQ |
| | | | | | ID NO: 4) |
| | | | Reverse | EX45-R | CAGCAGCCAGTGC |
| | | | | | CATAGTA (SEQ |
| | | | | | ID NO: 5) |
| 4 | CD19- | J13 | Forward | J13-F | GCCTCCTCTTCTT |
| | junct1-3 | | | | CCTCCTCTT |
| | | | | | (SEQ ID NO: 6) |
| | | | Reverse | J13-R | CCGGAACAGCTCC |
| | | | | | CCTTCCACCTTC |
| | | | | | (SEQ ID NO: 7) |
| 5 | HPRT1 | H | Forward | H-F | TGACACTGGCAAA |
| | | | | | ACAATGCA (SEQ |
| | | | | | ID NO: 8) |
| | | | Reverse | H-R | GGTCCTTTTCACC |
| | | | | | AGCAA (SEQ ID |
| | | | | | NO: 9) |

S3, obtaining CT values of multiple duplicates of an amplification curve of each of the amplification primer pairs according to an effective amplification result of the qRT-PCR, and removing reactions with a CT value of greater than 35; averaging the CT values of the multiple duplicates of the amplification curve of each of the amplification primer pairs, calculating Act according to a formula II with an internal reference HPRT1 or an amplification curve of CD19exon3-4 as a control; and calculating a level of a target CD19 mRNA by a 2-Δct relative quantification method according to formulas III and IV; wherein
$$MEANct ( X ) = ct ( duplicate 1 X ) + ct ( duplicate 2 X ) + … + ct ( duplicate nX ) / n , \qquad formula I$$
$$Δ ct ( target mRNA ) = MEANct ( target mRNA ) - MEANct ( internal reference ) , \qquad formula II$$
$$relative expression level ( fold - change ) = 2 \hat{} ( - Δ ct ( target mRNA ) ) , and \qquad formula III$$
$$MEANct ( target mRNA ) = ct ( duplicate 1 target RNA ) + ct ( duplicate 2 target RNA ) + … + ct ( duplicate n target RNA ) ) / n . \qquad formula IV$$

5. The method for detecting an expression state of CD19 in a tumor of a patient underwent an Anti-CD19 CAR-T cell therapy according to claim 4, wherein step S3 further comprises: relatively quantifying results of qRT-PCR based on the level of the target CD19 mRNA to determine a proportion of expression levels of different alternatively spliced isoforms of CD19 mRNA; and
the qRT-PCR in step S2 comprises: 1) initial denaturation at 95° C., 2) denaturation at 95° C. and annealing at 60° C., 40 cycles, 3) melt curve, denaturation at 95° C., annealing at 58° C. to 62° C., and denaturation at 95° C.

6. The method for detecting an expression state of CD19 in a tumor of a patient underwent an anti-CD19 CAR-T cell therapy according to claim 4, wherein the reaction system in step S2 comprises: the primer pair J13 with a final concentration of 200 nM, the primer pair EX34 and the primer pair EX45 each with a final concentration of 100 nM, and the cDNA stock solution with a final concentration of 100 ng/μL.

7. The method for detecting an expression state of CD19 in a tumor of a patient underwent an anti-CD19 CAR-T cell therapy according to claim 5, further comprising the following steps:
S4, mixing a CD19exon1-4 amplification primer pair in the reaction system to obtain a mixture, centrifuging the mixture, aliquoting the mixture into a tube strip, and adding a template cDNA in each tube of the tube strip to allow PCR amplification; wherein
sequences of the CD19exon1-4 amplification primer pair are:
FP:
(SEQ ID NO: 14)
GGAGAGTCTGACCACCATGC,
RP:
(SEQ ID NO: 15)
GGACACAGAGTCAGGGGGTA;
the reaction system comprises: the CD19exon1-4 amplification primer pair with a final concentration of 400 nM, 2 μL to 5 μL of the cDNA stock solution synthesized in step S1 as the template cDNA, and a 2×TaqPlusMasterMix DNA polymerase; and
the PCR amplification comprises: initial denaturation at 95° C.; denaturation at 95° C., annealing at 58° C. to 62° C., and extension at 72° C., 35 cycles; and extension at 72° C.; and
S5: subjecting a product obtained from the PCR amplification in step S4 to 1.5% agarose gel electrophoresis, determining a band and cutting a gel containing the band to allow purification, conducting bidirectional first-generation sequencing using the forward amplification primer and the reverse amplification primer in S2, and analyzing an obtained result.

8. The method for detecting an expression state of CD19 in a tumor of a patient underwent an anti-CD19 CAR-T cell therapy according to claim 4, wherein the reaction system in step S2 further comprises primer pair P and primer pair E

| | Amplific- | Primer | Orient- | | |
|---|---|---|---|---|---|
| Serial | ation | pair | ation of | Primer | Sequence |
| Number | region | name | primer | name | information |
| 6 | hPAX5 | P | Forward | P-F | GGGAGATCAGGGA |
| | | | | | CCGGC (SEQ ID |
| | | | | | NO: 10) |
| | | | Reverse | P-R | GCTGTGACTGGAA |
| | | | | | GCTGGGAC (SEQ |
| | | | | | ID NO: 11) |
| 7 | hEBF1 | E | Forward | E-F | TGCCGAGTCTTGC |
| | | | | | TCACAC (SEQ |
| | | | | | ID NO: 12) |
| | | | Reverse | E-R | CATTGACTGTCGT |
| | | | | | AGACACCAC |
| | | | | | (SEQ ID NO: |
| | | | | | 13) |

9. The method for detecting an expression state of CD19 in a tumor of a patient underwent an anti-CD19 CAR-T cell therapy according to claim 8, wherein the reaction system in step S2 comprises the primer pair P, the primer pair E, and the primer pair J13 each with a final concentration of 200 nM.
